(19) United States
(12) Patent Application Publication
(10) Pub. No.: US 2004/0171834 A1
(43) Pub. Date: Sep. 2, 2004

(54) Methods for producing hydroxyalkyl tropane esters

(76) Inventors: Anita H. Lewin, Chapel Hill, NC (US); James P. Hayes, Durham, NC (US); Desong Zhong, Cary, NC (US)

Correspondence Address: FISH & NEAVE, 1251 AVENUE OF THE AMERICAS, 50TH FLOOR, NEW YORK, NY, 10020-1105, US

(21) Appl. No.: 10/646,284

(22) Filed: Aug. 21, 2003

Related U.S. Application Data
(60) Provisional application No. 60/405,433, filed on Aug. 21, 2002.

(51) Int. Cl. C07D 451/04

(52) U.S. Cl. 546/129

(57) ABSTRACT

This invention provides a method for preparing a hydroxyalkyl tropane ester, comprising: (a) contacting a tropane and 1,1′-carbonyldiimidazole to produce an activated tropane ester; (b) contacting the activated tropane ester with an excess of an alkanediol to form a reaction mixture; and (c) maintaining the reaction mixture at a temperature and for a sufficient time for the activated tropane ester to react with the alkanediol to form the corresponding hydroxyalkyl tropane ester. This method may be used to produce hydroxyalkyl derivatives of tropanes such as benzoylecgonine, ecgonine and ecgonidine.

### CROSS REFERENCE TO RELATED APPLICATIONS

This application claims benefit under 35 U.S.C. § 119(e) of U.S. Provisional Application No. 60/405,433, filed Aug. 21, 2002, the disclosure of which is incorporated herein by reference.

### TECHNICAL FIELD OF INVENTION

This invention relates to novel synthetic chemical methods for producing hydroxyalkyl tropane esters.

### BACKGROUND

There are a number of synthetic methods reported in the literature for producing hydroxyalkyl esters. The most common methods include direct esterification of the corresponding acid or conversion of the acid to an acid halide (with reagents such as SOCl₂) followed by esterification. Other esterification methods use coupling agents such as dicyclohexyl carbodiimide (DCC) and dimethylaminopyridine (DMAP). In the case of 2-hydroxy esters, ring opening of an epoxide is also a common synthetic approach. We have found that none of these methods is ideal for producing hydroxyalkyl tropane esters because of poor yield, expense and the production of byproducts that are not easily removed from the desired final product (among other difficulties). This is particularly true of 2-hydroxypropyl tropane esters and regioisomers thereof.

A number of hydroxyalkyl tropane esters have useful biological properties or are useful as intermediates for producing compounds having biological activity. For example, certain hydroxypropyl tropane esters are active against several important diseases and disorders (see, for example, U.S. Pat. Nos. 5,376,667; 5,559,123 and 5,663,345, each of which is hereby incorporated herein in its entirety). The hydroxypropyl esters of benzoylecgonine, ecgonine, and ecgonidine are particularly useful. Examples of such esters include (without limitation) 2-hydroxypropyl ecgonidine, 1-hydroxy-2-propyl ecgonidine, 2-hydroxypropyl benzoylecgonine, 1-hydroxy-2-propyl benzoylecgonine, 2-hydroxypropyl ecgonine, and 1-hydroxy-2-propyl ecgonine. Methods for producing compositions comprising these hydroxypropyl tropane esters have been described in U.S. Pat. No. 5,376,667. The preferred method described in U.S. Pat. No. 5,376,667 utilizes the step of heating cocaine base in a propylene glycol/water solution (95% propylene glycol/5% water w/w) at 50° C. for 12 days, after which time less than 0.1% of the cocaine base starting material remained (see column 7, lines 3-17). The composition produced by this method comprises approximately 5% w/w of an active component mixture in propylene glycol, wherein the active component mixture comprises approximately 65% benzoylecgonine, 2% ecgonidine and 5% and 6%, respectively, of the 2-hydroxypropyl derivatives of benzoylecgonine and ecgonidine. It is difficult to isolate the hydroxypropyl tropane esters from this mixture in acceptable yield.

Particular methods for producing tropane esters of simple alcohols have been described (see, for example, Lewin, A. H.; Gao, Y.; Abraham, P.; Boja, J. W.; Khuar, M. J.; Carroll, F. I. J. Med. Chem., 1992, 35(1), 135-140). A variety of methods for producing 1,2-propanediol esters have also been reported. In general, direct esterification of 1,2-propanediol usually results in a mixture of primary and secondary monoesters, accompanied by varying amounts of diester, as described below in Scheme 1. In addition, secondary esters of 1,2-propanediol are known to have a propensity to rearrange to the primary esters (Cohen, T., Dughi, M., Notaro, V. A., Pinkus, G. J. Org. Chem. 1962, 27, 814).

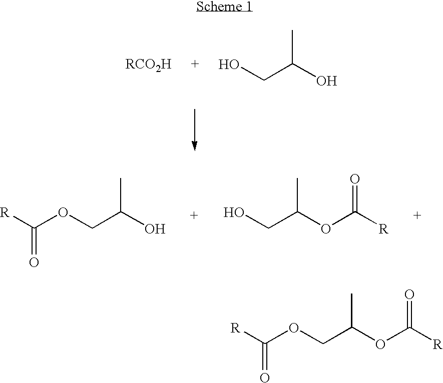

Esters produced from chiral substrates introduce the possibility of multiple stereoisomers of each regioisomer (for instance, in the case of the ecgonidine, benzoylecgonine and ecgonine esters produced from natural (R)-cocaine, there are RR and RS primary esters and RR and RS secondary esters).

In our laboratory, unsatisfactory results were obtained when we attempted to synthesize various hydroxypropyl tropane esters by numerous known techniques, including use of DMAP/pyridine, DMAP/DCC or DMAP/CDI, with stoichiometric amounts of acid and diol, as well as with excess diol. Some of these failed experiments are summarized below:

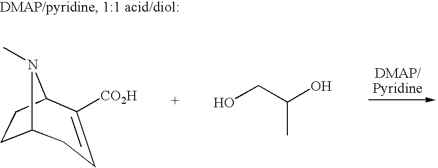

Attempted preparation of a solution of ecgonidine hydrochloride (1 g, 0.0049 mol), 1,2-propanediol (0.36 mL, 0.37 g, 0.0049 mol) and DMAP (30 m, 0.25 mmol) in pyridine (10 mL) resulted in precipitation of ecgonidine. Addition of acetonitrile (5 mL) provided a clear solution. No product was formed after stirring for 24 hours. Overnight reflux did not lead to product. Concentration of the solution under N₂, with heating, also failed to lead to significant product.

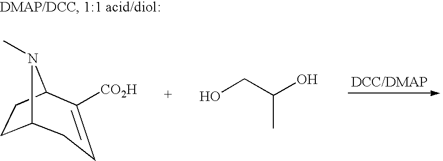

To a solution of ecgonidine hydrochloride (1 g, 0.0049 mol), 1,2-propanediol (0.36 mL, 0.37 g, 0.0049 mol) and DMAP (30 mg, 0.25 mmol) in DMF (20 mL) was added gradually DCC (1.11 g, 0.0054 mol). Stirring under N₂ soon resulted in a precipitate. After stirring at ambient temperature overnight the mixture was worked up to afford 1.64 g of a yellow-brown viscous gum. Column chromatography gave 0.56 g (40% yield) of the ester mixture, contaminated with 5% DMAP and 15% DCU.

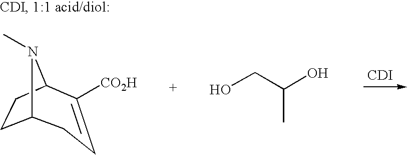

A solution of ecgonidine hydrochloride (1 g, 0.0049 mol) and CDI (0.80 g, 0.0049 mol) in DMF (20 mL) was stirred at ambient temperature under N₂ for 2 hrs and 1,2-propanediol (0.36 mL, 0.37 g, 0.0049 mol) was added. After stirring under N₂ at ambient temperature overnight the mixture was worked up to afford 0.53 g of a brown syrup consisting of mono- and di-esters of ecgondine and propanediol. Column chromatography gave 0.12 g (6.5%) of pure diester, 0.194 g of pure monoester (17% yield) and 0.29 g of the mono- and di-ester mixture.

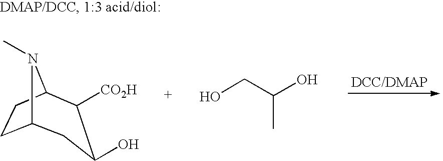

To an ice-cold solution of ecgonine hydrochloride (1 g, 0.0045 mol), 1,2-propanediol (0.99 mL, 00135 mol) and DMAP (30 mg, 0.25 mmol) in DMF (20 mL) was added gradually DCC (1.02 g, 0.0050 mol). After stirring at ambient temperature overnight the mixture was worked up to afford 1.15 g of an off-white solid. ¹H NMR showed the presence of the product, heavily contaminated with DCU and DMAP. Repeated purification attempts failed to remove these impurities and caused decomposition (e.g. elimination to give ecgonidine products).

These and other reported syntheses do not adequately address the need for a convenient method for producing individual hydroxyalkyl tropane esters easily and inexpensively, with good purity and in high yield. Accordingly, until the methods of this invention were discovered, there remained a need for improved methods to produce hydroxyalkyl tropane esters.

### SUMMARY

The invention described herein fulfills the need described above. In one embodiment, this invention provides a method for preparing a hydroxyalklyl tropane ester, comprising:

(a) contacting a tropane and 1,1′-carbonyldiimidazole to produce an activated tropane ester;

(b) contacting the activated tropane ester with an excess of an alkanediol to form a reaction mixture; and

(c) maintaining the reaction mixture at a temperature and for a sufficient time for the activated tropane ester to react with the alkanediol to form the corresponding hydroxyalkyl tropane ester.

The details of one or more embodiments of the invention are set forth in the description below. Other features, objects, and advantages of the invention will be apparent from the description and claims that follow.

### DETAILED DESCRIPTION

As used herein:

The term “alkyl” (whether used alone or in combination with other terms) refers to a saturated straight chain or branched chain, primary, secondary, or tertiary hydrocarbon radical. In one embodiment of this invention, the alkyl is a C₁-C₁₈ alkyl radical, in another embodiment a C₁-C₁₀ alkyl radical, and in yet another embodiment a C₁-C₆ alkyl radical, including, without limitation, methyl, ethyl, propyl, butyl, pentyl, hexyl, isopropyl, isobutyl, sec-butyl, t-butyl, isopentyl, amyl, and t-pentyl. For the purposes of this invention, any carbon in the alkyl segment may be substituted with oxygen (O), sulfur (S), or nitrogen (N). Further, alkyl segments may optionally be substituted with one or more conventionally used alkyl substituents, such as amino, alkylamino, alkoxy, alkylthio, oxo, halo, acyl, nitro, hydroxyl, cyano, aryl, alkylaryl, aryloxy, arylthio, arylamino, carbocyclyl, carbocyclyloxy, carbocyclylthio, carbocyclylamino, heterocyclyl, heterocyclyloxy, heterocyclylamino, heterocyclylthio, and the like. Unsubstituted alkyls are included as an embodiment of this invention. Propyl is included in another embodiment of this invention.

The term “alkanediol” refers to an alkyl moiety comprising two hydroxyl groups located at any position on the alkyl chain. In one embodiment, the alkanediol is 1,2-propanediol. It should be noted that in some cases, more than two hydroxyl groups may be present on the alkyl chain.

The term “benzoylmethylecgonine” or “BME” refers to the chemical entity 3-benzoyloxy-2-carbomethyoxy-8-methyl-8-azabicyclo[3.2.1]octane. BME can exist in four diastereomeric forms (cocaine, pseudococaine, allococaine and allopseudococaine) and each diastereomer has two optical antipodes. Any one of these compounds or any combination of more than one of these compounds is encompassed by the invention herein. BME is typically prepared as a salt (e.g., cocaine HCl) or a reduced base (e.g., cocaine alkaloid) according to known methods.

The term “CDI” refers to 1,1′-carbonyldiimidazole.

The term “DCC” refers to dicyclohexylcarbodiimide.

The term “DCU” refers to dicyclohexylurea.

The term “DMAP” refers to 4-dimethylaminopyridine

The terms “2-hydroxypropyl ester”, “2-hydroxypropyl ester derivatives”, “2-HP derivatives” and other similar terms used herein, refer to the 2-hydroxypropyl ester derivatives of tropane acids such as benzoylecgonine, ecgonine and/or ecgonidine. When these terms are used in general herein, they are meant to refer to any of these 2-hydroxypropyl ester derivatives.

The term “substantially all”, when referring to the reactions of this invention, means that more than approximately 80% of the tropane starting material has reacted. In one embodiment, more than approximately 85%, and in another embodiment, more than approximately 90% and in yet another embodiment, more than approximately 95% of the tropane starting material has reacted. The progress of such reactions may be monitored by thin layer chromatography (TLC), high pressure liquid chromatography (HPLC) and other means known to those of ordinary skill in the art.

The term “tropane” refers to a compound having a tropane ring, including without limitation benzoylecgonine, ecgonidine and ecgonine.

This invention provides a method for preparing an hydroxyalkyl tropane ester, comprising:

(a) contacting a tropane and 1,1′-carbonyldiimidazole to produce an activated tropane ester;

(b) contacting the activated tropane ester with an excess of an alkanediol to form a reaction mixture; and

(c) maintaining the reaction mixture at a temperature and for a sufficient time for the activated tropane ester to react with the alkanediol to form the corresponding hydroxyalkyl tropane ester.

The method of this invention advantageously produces hydroxyalkyl tropane esters in good yield and free from impurities that complicate or prevent effective purification of the final product. The first steps of the reaction of this invention comprise reacting a tropane acid and 1,1′-carbonyldiimidazole to form an activated tropane ester, followed by reacting the tropane ester with an excess amount of alkanediol to form a reaction mixture. The tropane acid may be added as the free acid or as a salt, such as an acid addition salt (such as a hydrochloride salt). For example, in the case of ecgonine and ecgonidine, their respective hydrochloride salts may be used as the tropane in this reaction. In one embodiment of this invention, the first two steps can advantageously be performed without purification of the activated tropane ester. In a particular embodiment of the invention, the tropane is the free acid of benzoylecgonine, ecgonidine or ecgonine or a salt thereof, and the alkanediol is 1,2-propanediol. The reaction may be carried out in any suitable organic solvent, including (without limitation) methylene chloride and dimethylformamide (DMF). The reaction may optionally be carried out under an inert gas, such as N₂. Typically, the tropane is contacted with CDI for between 1 minute and 36 hours (after which time, a suspension may be formed and gas evolution may be observed) to form the activated tropane ester of step (a).

The reaction mixture is then formed by contacting the activated tropane ester with an excess amount of the appropriate alkanediol. In particular embodiments of this invention, the excess amount is at least about 2, 2.5 or 3 equivalents to 1 equivalent of tropane. The solution can be stirred or otherwise agitated to promote a steady and efficient reaction.

The reaction mixture should be maintained at a temperature and for a sufficient time for the activated tropane to react with the alkanediol and form the corresponding hydroxyalkyl tropane ester. In one embodiment of this invention, the temperature of the reaction is maintained at between about 0° C. and the boiling point of the solution. For example, the reaction may be run at ambient temperature. The reaction can be monitored to determine when substantially all of the tropane starting material has reacted. The reaction is ordinarily carried out for between about 1 hour and 5 days and in a particular embodiment of this invention, between about 5 hours and 2 days. The amount of tropane starting material remaining in the reaction mixture can be monitored during the course of the reaction using known techniques, such as gas chromatography, high performance liquid chromatography (HPLC), thin layer chromatography (TLC) and/or mass spectrophotometry.

In an additional embodiment of this invention, the hydroxyalkyl tropane ester can be further isolated or otherwise purified from the reaction mixture. To isolate the final product (for example, after substantially all of the tropane starting material has reacted), the reaction mixture may be filtered (if solid particles have formed) then the final product may be extracted (including by solid phase extraction) or otherwise isolated from the reaction mixture. Depending on the nature of the desired product and other components of the reaction, other means of isolation and purification that may be used include (without limitation) crystallization and chromatography (such as by TLC or HPLC). In the case of final products that are not solids (e.g., oils or gums), it may be convenient to form solid salts that can then be crystallized. In any event, additional purification steps may be employed to further enhance the purity of the final product. Such further purification may involve column chromatography or other suitable techniques known to those of ordinary skill in the art.

### EXAMPLES

The following specific examples are to be construed as merely illustrative, and not limitative of the disclosure in any way.

Thin layer chromatography was carried out using EM Science silica gel 60 or RP18 TLC plates; visualization was under UV or in an iodine chamber, as appropriate. ¹H NMR spectra were obtained on either a Bruker DPX-300 or a Bruker AMX 500 spectrometer. HPLC analysis was carried out using Dynamax Solvent Delivery System Model SD-300, a Rheodyne 7725I injector and a Dynamax Absorbance Detector Model UV-1 or a Sedex Model 75 Evaporative Light Scattering Detector. The ecgonidine, ecgonine and benzoylecgonine acids used as tropane starting material for the methods of this invention can be obtained from a commercial source or alternatively, produced from cocaine by known methods, such as those exemplified herein.

### Example 1

Production of Hydroxypropyl Esters of Ecgonidine

1.1. Ecgonidine Hydrochloride

A solution of cocaine hydrochloride (15.0 g, 0.044 mol) in conc. HCl (75 mL) was refluxed overnight in a round bottomed flask. After cooling to room temperature the precipitated benzoic acid was removed by filtration and the filtrate was washed with Et₂O (3×25 mL). The aqueous phase was evaporated to a small volume, treated with charcoal and evaporated further. The residue was crystallized from acetone. After a second recrystallization, 6.7 g (65%) of white crystals was collected: m.p. 245-248° C.; [α]_{D}²³−67° (c 1, H₂O).

1.2. 2-Hydroxypropyl Ecgonidine and 1-Hydroxy-2-Propyl Ecgonidine

A solution of ecgonidine hydrochloride from Example 1.1(5 g, 25 mmol) and 1,1 ′-carbonyldiimidazole (CDI) (4 g, 25 mmol) in dry DMF (50 mL) was stirred under N₂. After 10 min a suspension was formed and gas evolution was observed. The reaction mixture was treated with excess 1,2-propanediol (5.5 mL, 75 mmol) and stirring was continued. After 2 days the mixture was filtered and the white solid was washed with CH₂Cl₂. The combined filtrate and washings was concentrated under vacuum and the residual brown oil was dried in vacuo overnight. The oil was partitioned between CH₂Cl₂ (100 mL) and 20% NH₄OH (50 mL). The organic phase was washed twice more with 20% NH₄OH (50 mL), then dried over Na₂SO₄, concentrated and dried in vacuo (3.30 g). This material was purified by column chromatography on SiO₂ (350 g), eluting with CHCl₃:MeOH:NH₄OH (90:10:1). A total of 1.44 g (25%) of pure material was collected. Another 1.1 g (19.6%) of somewhat less pure material was also recovered.

1.3. HPLC Analysis

Analysis of the hydroxypropyl ecgonidine esters was carried out as follows:

Column: Waters Xterra MS C 18 (3.9*150 mm, 5 μm)

Solvents: A: 0.1% TFA-H₂O, B: CH₃OH; 3% B; 0.5 mL/min

Detection: 210 nm

The retention times were:

Rt (min): (RR)-2-hydroxypropyl-ecgonidine 34.2; (RR)-1-hydroxypropyl ecgonidine 41.8; (SR)-1-hydroxypropyl ecgonidine 32.0; (SR)-2-hydroxypropyl-ecgonidine 32.0

1.4. NMR

The tropane portion of the proton NMR spectra (300 MHz, DMSO-d₆) of the 4 esters were indistinguishable from each other. The chemical shifts, δ (ppm), are: 1.41, 1.67 (2H, AB, H-6,7), 1.77, 1.84 (1H, AB, H-4e), 1.98 (2H, m, H-6,7), 2.19 (3H, s, CH₃), 2.509 (1H, m, H-4a), 3.10 (1H, m, H-5), 3.57 (1H, m, H-1), 6.73 (minor), 6.79 (major) (1H, m, H-3).

The proton NMR spectrum of hydroxypropyl portion of the (SR)-2-hydroxypropyl ecgonidine (300 MHz, DMSO-d₆), δ (ppm) follows: 1.07 (3H, d, J=6.0 Hz), CH₃), 3.86 (1H, m, J=6.0 Hz, CH), 3.91 (2H, AB, CH₂). For (RR)-2-hydroxypropyl ecgonidine: 1.07 (3H, d, J=6.3 Hz, CH₃), 3.84 (1H, m, CH), 3.90 (2H, m, CH₂). For (SR)-1-hydroxy-2-propyl ecgonidine: 1.13 (3H, d, J=6.3 Hz, CH₃), 3.44 (2H, AB, CH₂), 4.81 (1H, m, J=6.0 Hz, CH). For (RR)-1-hydroxy-2-propyl ecgonidine: 1.14 (3H, d, J=6.3 Hz, CH₃), 3.594 (2H, AB, J=6.0 Hz, CH₂), 4.82 (1H,m, J=6.0 Hz, CH).

### Example 2

Production of Hydroxypropyl Esters of Benzoylecgonine

2.1. Benzoylecgonine

Cocaine hydrochloride (17.0 g, 0.05 mol) was free-based with NH₄OH and extracted into CHCl₃. The combined CHCl₃ layers were dried over Na₂SO₄ and concentrated to afford a white solid. This material was dissolved in H₂O (30 mL) and dioxane (30 mL). The resulting mixture was stirred at 60° C. for seven days. The H₂O/dioxane was removed under reduced pressure yielding 12.5 g (86%) of a white solid: m.p. 198-199° C. {lit (86-92°) 195° C.; S. Budavari, Merck Index, Rahway, N.J., Monograph 1125, p.174 (1989)}; [α]_{D}²²−57° (c 6.1, 100% EtOH) {lit −45° (c 3, 100% EtOH); ibid}.

2.2. 2-Hydroxypropyl Benzoylecgonine and 1-Hydroxy-2-Propyl Benzoylecgonine

After stirring at ambient temperature for 24 hours, a solution of anhydrous benzoylecgonine (6.066 g, 21.0 mmol) and 1,1′-carbonyldiimidazole (3.406 g, 21.0 mmol) in CH₂Cl₂ (100 mL) was treated with 1,2-propanediol (10.2 mL, 10.6 g, 138.0 mmol). Stirring was continued as the progress of the reaction was monitored by HPLC. When ester formation was slowed the reaction mixture was diluted with CHCl₃ (100 mL) and extracted with 3N HCl (4×40 mL). The combined extract was cooled to 0° C., basified to pH 10 with NH₄OH, and extracted with CHCl₃ (5×40 mL). The combined extract was washed with H₂O, dried with Na₂SO₄, and concentrated. The residue was dried in vacuo overnight to a clear syrup (6.8 g, 94% yield).

2.3. HPLC Analysis

Analysis of the hydroxypropyl benzoylecgonine esters was carried as follows:

Column: Phenomenex Synergi Polar-RP (3*150 mm, 4 μm, 80A)

Solvents: A: 0.1% TFA-H₂O, B: CH₃OH; 30% B; 0.6 mL/min

Detection: 225 nm

The retention times were:

Rt (min): (RR)-2-hydroxypropyl benzoylecgonine 10.5; (RR)-1-hydroxy-2-propyl benzoylecgonine 12.6; (SR)-1-hydroxy-2-propyl benzoylecgonine 12.6; (SR)-2-hydroxypropyl benzoylecgonine 17.1

2.4. NMR

The tropane portion of the proton NMR spectra (300 MHz, DMSO-d₆) of the 4 esters were very similar. The chemical shifts, δ (ppm), are: 1.64 (2H, AB, H-6,7), 1.72 (1H, m, H-4e), 2.10 s (2H, m, H-6,7), 2.00 (3H, s, CH₃), 2.24 (1H, t, H-4a), 2.95, 2.98, 3.03 (1H, dd, H-2 for (RR)-2-hydroxypropyl benzoylecgonine and 1-hyrdoxy-2-propyl benzoylecgonine, (SR)-2-hydroxypropyl benzoylecgonine, and (SR)-1 -hydroxy-2-propyl benzoylecgonine, respectively) 3.03 (1H, m, H-5), 3.54 (1H, m, H-1), 5.13 (1H, m, J=6.0 Hz, H-3), 7.46 (2H, m, o-ArH), 7.57 (1H, m, p-ArH), 7.85 (2H, m, m-ArH).

The proton NMR spectrum of hydroxypropyl portion of the (SR)-2-hydroxypropyl benzoylecgonine (300 MHz, DMSO-d₆), δ (ppm) follows: 1.07 (3H, d J=6.0 Hz, CH₃), 3.78 (1H, m J=6.0 Hz, CH), 3.97 (2H, AB, CH₂). For (RR)-2-hydroxypropyl benzoylecgonine: 1.00 (3H, d (J=6.3 Hz), CH₃), 3.78 (1H, m, CH), 3.86 (2H, m, CH₂). For (SR)-1-hydroxy-2-propyl benzoylecgonine: 1.06 (3H, d (J=6.3 Hz), CH₃), 3.78 (2H, AB, CH₂), 4.90 (1H, m, (J=6.0 Hz), CH). For (RR)-1-hydroxy-2-propyl benzoylecgonine: 1.10 (3H, d (J=6.3 Hz), CH₃), 3.38 (2H, AB (J=6.0 Hz), CH₂), 4.83 (1H, m, (J=6.0 Hz), CH).

### Example 3

Production of Hydroxypropyl Esters of Ecgonine

3.1 Ecgonine Hydrochloride

(−)-Cocaine hydrochloride (25 g, 0.07 mol) was dissolved in H₂O (300 mL) in a 2 L three-necked round bottom flask and concentrated HCl (26 mL) was added. After 7 h reflux with stirring, under nitrogen, the reaction mixture was cooled to room temperature and left stirring under nitrogen overnight. The precipitated benzoic acid was removed by filtration and the filtrate was evaporated to a yellow paste. The solid obtained by crystallization from MeOH/Et₂O was washed thoroughly with Et₂O and dried (13.1 g, 0.06 mol, 86%). The m.p. was 246-247° C., {lit 246° C.}; [α]_{D}²³ −44.3° (c.1.52, H₂O) lit. −45.2 (0.5%, H₂O); M. R. Bell and S. Archer, J. Am Chem. Soc. 82, 4642-4644 (1960)}

3.2. 2-Hydroxypropyl Ecgonine and 1-Hydroxy-2-Propyl Ecgonine

A solution of ecgonine hydrochloride (4.43 g, 0.02 mol) and carbonyldiimidazole (3.24 g, 0.02 mol) in dry DMF (50 mL) was stirred under N₂. After 10 hours a suspension was formed and gas evolution was observed. The reaction mixture was treated with excess 1,2-propanediol (14.7 mL, 0.20 mol) and stirring was continued. After stirring overnight the mixture was concentrated under vacuum and the residual syrup was partitioned between CH₂Cl₂ (100 mL) and 20% NH₄OH (50 mL). The organic phase was washed twice more with 20% NH₄OH (50 mL), then dried over Na₂SO₄, concentrated and dried in vacuo (2.43 g). This material was purified by column chromatography on SiO₂ (325 g), eluting with CHCl₃:MeOH:NH₄OH (90:10:1). A total of 0.66 g (14%) of pure material was collected. Another 0.38 g (8%) of less pure material was also recovered.

3.3 NMR

The tropane portions of the proton NMR spectra (500 MHz, DMSO-d₆) of the 4 esters were indistinguishable from each other. The chemical shifts, δ (ppm), are: 1.51 (2H, AB, H-6, 7), 1.62 (1H, AB, H-4e), 1.85 (1H, m, H-4a), 1.90 (2H, m, H-6, 7), 2.10 (3H, s, CH₃), 271 (1H,m, H-2), 3.05 (1H, m, H-5), 3.55 (1H, m, H-1), 3.72 (1H, m, H-3).

The proton chemical shifts of hydroxypropyl portion of the diastereomers were indistinguishable. The assignments for the primary ester (2-hydroxypropyl ecgonine) were (500 MHz, DMSO-d₆), δ (ppm): 1.09 (3H, d, J=6.0 Hz, CH₃), 3.86 (1H, m, J=6.0 Hz, CH), 3.82 and 3.91 (2H, AB, CH₂). For the secondary ester (1-hydroxy-2-propyl ecgonine) the assignments were (500 MHz, DMSO-d₆), δ (ppm): 1.12 (3H, d, J=6.4 Hz, CH₃), 3.40 (2H, m, CH₂), 4.84 (1H, m, CH).

### Other Embodiments

A number of embodiments of the invention have been described. Nevertheless, it will be understood that various modifications may be made without departing from the spirit and scope of the invention. Accordingly, other embodiments are within the scope of the following claims.

What is claimed is:

1. A method for preparing a hydroxyalkyl tropane ester, comprising:
(a) contacting a tropane and 1,1′-carbonyldiimidazole to produce an activated tropane ester;
(b) contacting the activated tropane ester with an excess of an alkanediol to form a reaction mixture; and
(c) maintaining the reaction mixture at a temperature and for a sufficient time for the activated tropane ester to react with the alkanediol to form the corresponding hydroxyalkyl tropane ester.

2. The method according to claim 1, wherein the alkanediol is 1,2-propanediol.

3. The method according to claim 1, wherein the tropane is ecgonidine.

4. The method according to claim 3, wherein reaction of step (b) is carried out in dry DMF.

5. The method according to claim 3, wherein the excess of alkanediol is at least about 2 equivalents of alkanediol to 1 equivalent of tropane.

6. The method according to claim 3, wherein the reaction of step (b) is carried out under an inert gas.

7. The method according to claim 6, wherein the inert gas is nitrogen.

8. The method according to claim 1, wherein the tropane is ecgonine.

9. The method according to claim 7, wherein reaction of step (b) is carried out in dry DMF.

10. The method according to claim 7, wherein the excess of alkanediol is at least about 2 equivalents of alkanediol to 1 equivalent of tropane.

11. The method according to claim 7, wherein the reaction of step (b) is carried out under an inert gas.

12. The method according to claim 11, wherein the inert gas is nitrogen.

13. The method according to claim 1, wherein the tropane is benzoylecgonine.

14. The method according to claim 13, wherein reaction of step (b) is carried out in methylene chloride.

15. The method according to claim 13, wherein the excess of alkanediol is at least about 2 equivalents of alkanediol to 1 equivalent of tropane.

16. The method according to claim 1, further comprising the step of isolating the hydroxyalkyl tropane ester from the reaction mixture.

17. The method according to claim 16, wherein the isolation is performed by extraction.

18. The method according to claim 16, further comprising the step of purifying the isolated hydroxyalkyl tropane ester.

19. The method according to claim 18, wherein the purification is performed by column chromatography.
